(19) United States
(12) Patent Application Publication
(10) Pub. No.: US 2017/0042899 A1
(43) Pub. Date: Feb. 16, 2017

(54) Use of Tetrahydrobiopterine Derivatives in the Treatment and Nutrition of Patients With Amino Acid Metabolic Disorders

(71) Applicant: BioMarin Pharmaceutical Inc., Novato, CA (US)

(72) Inventors: Ania Muntau-Heger, Munich DEX (DE); Adelbert A. Roscher, Munich DEX (DE)

(21) Appl. No.: 15/211,180

(22) Filed: Jul. 15, 2016

Related U.S. Application Data
(63) Continuation of application No. 10/539,842, filed on Mar. 20, 2006, now abandoned.

(30) Foreign Application Priority Data
Dec. 20, 2002 (DE) 10260263.8

(51) Int. Cl. A61K 31/519; A23L 33/10

(52) U.S. Cl. CPC A61K 31/519; A23L 33/10; A23V 2002/00

(57) ABSTRACT

The invention relates to the use of tetrahydrobiopterine and the derivatives thereof in the production of a medicament to improve protein tolerance for the treatment of diseases arising from an amino acid metabolic disorder, e.g. hyperphenylalaninemia. The invention also relates to a composition which contains tetrahydrobiopterine or derivatives thereof in addition to a special mixture of amino acids. The invention can, for instance, be used as a food which is low in phenylalanine in the complete nutrition of hyperphenylalaninemic patients. Tests carried out within the text of said invention revealed that by treating the context of said invention revealed that by treating patients who had phenylalanine concentrations of more than 200 μmol/l in their blood with tetrahydrobiopterine, it was possible to reduce the concentrations of phenylalanine by 37% to 92%.

The present invention concerns the use of tetrahydrobiopterine derivatives according to claim 1, a composition according to claim 13, the use of tetrahydrobiopterine derivatives as nutritional supplements according to claim 26, a special food according to claim 28, a phenylalanine-poor special nutritional substance according to claim 40, as well as a diagnostic for diagnosis of tetrahydrobiopterine sensitive diseases which are associated with disrupted amino acid uptake according to claim 43.

Diseases caused by amino acid uptake disturbances are generally relatively widely disseminated diseases which are most commonly attributable to genetics. As pathophysiological correlate one may identify reduced activities of certain enzymes with a consequence of elevated or lowered concentrations of amino acids and the therefrom synthesized neurotransmitters and second messengers, as well as disrupted tolerance (protein tolerance) of certain amino acid components in the diet.

For the purposes of the present invention the term “afflictions as a result of a disrupted amino acid uptake” shall be understood to include the following pathophysiological conditions:

Conditions with elevated phenylalanine or reduced tyrosine, serotonin or dopamine in body fluids, tissues or cells, in particular in conditions with reduced phenylalanine hydroxylase, tyrosinhydroxylase, tryptophanhydroxylase and NO-Synthase activity. These conditions can—without however being limited thereto—include the following phases of disease: phenylketonurea, in particular mild phenylketonurea, classical phenylketonurea; pigment disruptions of the skin, in particular vitiligo; as well as conditions caused by reduced cellular access to catecholamines, in particular orthostatic hypotension (Shy-Drager Syndrome), muscular dystonia; as well as neurotransmitter disturbances, in particular Schizophrenia; conditions caused by reduced cellular access to dopamine or serotonin as consequence of tyrosinhydroxylase or tryptophanhydroxylase deficit, in particular Parkinson's disease, depressive diseases as well as dystonia movement incapacitance (torsion dystonia), conditions of reduced NO-synthase activity, in particular endothelial dysfunction, reduced resistance to infection.

One known interference of amino acid metabolism, which is based upon the lack of or reduced ability to metabolize phenylalanine, is hyperphenylalaninemia, which is brought about by a lack of phenylalanine hydroxylase. At least one half of the afflicted patients manifest with mild clinical phenotypes. The single possible treatment in accordance with the state of the art of most amino acid metabolism diseases, such as for example hyperphenylalaninemia, lies therein, to nurture the patients with a diet which contains products which do not contain the amino acids associated with the special metabolic disturbance or, as the case may be, only contain these in small amounts.

The hyperphenylalaninemia was one of the first genetic diseases, which could be treated. In most cases hyperphenylalaninemia was caused by a lack of phenylalinhydroxylase, brought about by mutations on the phenylalinhydroxylase genes. The therewith associated phenotypes range, in their degree of affliction, from the classical phenylketonurea (Online Mendelian Inheritance Genetics in Humans number 261600) (Online Mendelian Inheritance in Man number 261600) up to mild phenylketonurea and mild hyperphenylalaninemia. At least half of the concerned patients suffer from one of the milder clinical phenotypes. Both patients suffering from a classical phenylketonurea as well as patients suffering from a mild phenylketonurea must be careful their entire lives to partake of a protein-poor diet, in order avoid neurologic consequences and to ensure normal cognitive development, in comparison to which patients with a mild hyperphenylalaninemia in certain cases require no treatment. In conjunction with the very strict diet there is the risk of a nutrient-associated deficiency symptom and it imposes a heavy burden for the patients and their families.

A causal effective therapy does not exist until know in the state of the art, so that for the concerned patients no other possibility exists, than to maintain the strict diet, if they do not wish to risk substantial consequential symptoms of the amino acid metabolic disturbances and, for example, the therewith associated hyperphenylalaninemia. The neurological consequential symptoms include for example irreversible damage of the nerve system and the brain, mental retardation, all the way to imbecility. Beyond this, kidney damage, liver damage and damage of the sensory organs has been described.

For the concerned patients this means—for example in the case of hyperphenylalaninemia—that one must supply these with a phenylalanine poor diet. Since phenylalanine is an important protein building block, in particular in the animal world, it is naturally difficult to feed patients with amino acid metabolic disorders—without provocation of undesired and toxic phenylalanine increases. Beyond this, diet related deficiency symptoms can occur.

For this, previously amino hydrolysate was employed in the state of the art, which could be produced from phenylalanine low proteins by acid or alkalide hydrolysis.

This type of product had a more than bad taste, and was frequently unbearable for patients in the long term. Besides these hydrolysates, there only came into consideration, depending upon appropriate dietetic concept, very selective foods, mostly of vegetarian nature, as nutrients for the afflicted patients.

In comparison, the synthetic amino acid mixtures which do not contain the specific amino acids with which the metabolic disturbance is concerned, already exhibited a strong improvement in comparison to the traditional hydrolysates.

Phenylalanine free products on this basis are known for example from U.S. Pat. No. 5,393,532, and have until now been used as special nutrients for hyperphenylalaninemia phenylketonurea patients.

It is further known from WO 98/08402 A1, to produce special nutrients on the basis of casein-glyko-macropeptides in conjunction with amino acid mixtures, in order to feed patients in need thereof, for example, free of phenylalanine.

With regard to taste these amino acid mixtures are however much below the level of conventional nutrients.

In summary it can be concluded that a strict diet plan to be maintained lifelong, which is tailored to a specific amino acid metabolic disturbance, represents a strong psychosocial burden, and that other treatment methods have until now not been successful.

Beginning with the state of the art it is the task of the present invention to make available materials which can be employed, on the one hand, in the framework of a therapeutic treatment of amino acid metabolic disturbances, and on the other hand, can be employed for the production of nutrient means, in particular dietetic special nutrients for amino acid metabolic disturbance afflicted patients.

The above task is solved by the use of tetrahydrobiopterine derivatives according to claim 1, a composition according to claim 13, a use of tetrahydrobiopterine derivatives as nutrient supplement according to claim 26, a special nutrient according to claim 28 as well as a phenylalanine poor special nutrient means according to claim 40.

It is a further task of the present invention to make available a diagnostic for such amino acid metabolism disturbances, which can be beneficially influenced or enhanced by tetrahydrobiopterine derivatives.

This task is solved by a diagnostic according to claim 43.

In particular the present invention concerns the use of at least one compound with the following general formula:

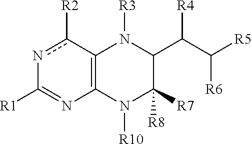

wherein R1 is selected from the group consisting of: H, OH, SH, F, Cl, Br, I, NH₂, N(CH₃)₂, N(C₂H₅)₂, N(C₃H₇)₂; NH-Acyl, wherein the Acyl residue contains one to 32 carbon atoms, in particular CH₃O, preferably 9 to 32, preferably 9 to 20 carbon atoms;
wherein R2 is selected from the group consisting of: H, OH, SH, NH₂, F, Cl, Br, I, O, S;
wherein R3 is selected from the group consisting of: H, CH₃, C₂H₅;
wherein R4 and R6 selected independent of each other are from the group consisting of: H, OH, SH, NH₂, F, Cl, Br, I, acetyl, OX, wherein X is a C1 to C32 acyl residue, in particular a C9 to a C32 acyl residue, preferably a C9 to C20 acyl residue, wherein R5 is selected from the group comprised of: phenyl, CH₃, C₂H₅, C₃H₇, butyl, isobutyl, t-butyl;
wherein R7 and R8 are selected independent of each other from the group consisting of: H, OH, SH, NH₂, F, Cl, Br, I, CH₃, COOH, CHO, COOR9, wherein R9 CH₃, C₂H₅, C₃H₇, butyl;
wherein R10 is selected from the group consisting of: H, CH₃, C₂H₅, and wherein - - represents an optional double bond; as well as
their pharmaceutically acceptable salts;
for producing a medicament for improving protein tolerance for treatment of diseases as a consequence of a disrupted or impeded amino acid metabolism.

In the following preferred embodiments of the inventive use are described:

Particularly suited for the inventive use is a compound, selected from the group consisting of 5,6,7,8-tetrahydrobiopterine, sapropterin, in particular their hydrochlorides or sulfates, as well as a compound with the following structure:

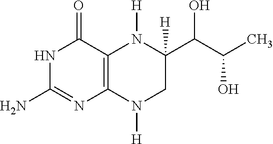 [SMILES: C[C@H](O)C(O)[C@H]1CNc2nc(N)[nH]c(=O)c2N1]

(−)-(1′R,2′S,6R)-2-amino-6-(1′,2′-dihydroxypropyl)-5,6,7,8-tetrahydro-4(3H)-pteridinone,
   in particular their dihydrochloride; and/or
2-N-stearoyl-1′,2′-di-O-acetyl-5,6,7,8-tetrahydrobiopterine; and/or
2-N-decanoyl-1′,2′-di-O-acetyl-5,6,7,8-tetrahydrobiopterine; and/or
2-N-palmitoyl-1′,2′-di-O-acetyl-5,6,7,8-tetrahydrobiopterine; and/or
2-N-linoleoyl-1′,2′-di-O-acetyl-5,6,7,8-tetrahydrobiopterine.

As salts, in particular hydrochlorides or sulfates can be employed.

The above mentioned compounds can in particular be employed as medicaments for treatment of the following diseases or, as the case may be, amino acid metabolism disturbances:

Conditions with elevated phenylalanine or reduced tyrosin in body fluids, tissues or cells, in particular conditions with reduced phenylalanine hydroxylase activity; phenylketonurea, in particular mild phenylketonurea, classical phenylketonurea; pigment disturbances of the skin, in particular vitiligo; conditions caused by reduced cellular access to catecholamine, in particular orthostatic hypotension (Shy-Drager Syndrome), muscular dystonia; as well as neurotransmitter disturbances, in particular schizophrenia.

Preferably, as the pharmaceutically acceptable salt, a hydrochloride, in particular a dihydrochloride, is employed.

Beyond this, a refinement can be made to the present invention if one employs at least one compound with the following general formula as chaperone, in particular chemical chaperone, or so called protein-folding aid:

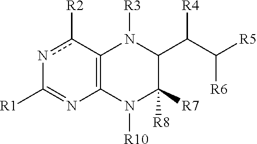

wherein R1 is selected from the group consisting of: H, OH, SH, F, Cl, Br, I, NH₂, N(CH₃)₂, N(C₂H₅)₂, N(C₃H₇)₂; NH-acyl, wherein the acyl residue contains 1 to 32 carbon atoms, in particular CH₃O, preferably 9 to 32, preferably 9 to 20 carbon atoms;
wherein R2 is selected from the group consisting of H, OH, SH, NH₂, F, Cl, Br, I, O, S;
wherein R3 is selected from the group consisting of: H, CH₃, C₂H₅;
wherein R4 and R6 are selected independently of each other from the group consisting of: H, OH, SH, NH₂, F, Cl, Br, I, acetyl, OX, wherein X is a C1 to C32 acyl residue, in particular a C9 to C32 acyl residue, preferably a C9 to C20 acyl residue;
wherein R5 is selected from the group consisting of: phenyl, CH₃, C₂H₅, C₃H₇, butyl, isobutyl, t-butyl;
wherein R7 and R8 are selected independent of each other from the group consisting of: H, OH, SH, NH₂, F, Cl, Br, I, CH₃, COOH, CHO, COOR9, wherein R9 CH₃, C₂H₅, C₃H₇, butyl;
wherein R10 is selected from the group consisting of: H, CH₃, C₂H₅, and -- represents an optional double bond; as well as their pharmaceutically acceptable salts. Also in the use as chaperone it is preferred when the compound is selected from the group consisting of 5, 6, 7, 8-tetrahydrobiopterine, sapropterin, in particular their hydrochlorides, as well as the compound with the following structure:

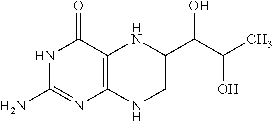 [SMILES: CC(O)C(O)C1CNc2nc(N)[nH]c(=O)c2N1]

(−)-(1′R,2′S,6R)-2-amino-6-(1′,2′-dihydroxypropyl)-5,6,7,8-tetrahydro-4(3H)-pteridinone,
   in particular their dihydrochlorides or sulfates and/or
2-N-stearoyl-1′,2′-di-O-acetyl-5,6,7,8-tetrahydrobiopterine; and/or
2-N-decanoyl-1′,2′-di-O-acetyl-5,6,7,8-tetrahydrobiopterine; and/or
2-N-palmitoyl-1′,2′-di-O-acetyl-5,6,7,8-tetrahydrobiopterine; and/or
2-N-linoleoyl-1′,2′-di-O-acetyl-5,6,7,8-tetrahydrobiopterine.

The mentioned compounds have demonstrated themselves to be exceptional for reducing protein misfolding and thereby for improvement of enzyme activity, in particular in structural anomalies of enzymes which require tetrahydrobiopterine as co-factor, for example, in defects of phenylalanine hydroxylase. By this mechanism of action these are advantageously suited for production of medicaments, which are suited for treatment of sources of illness which can be traced back to structural anomalies of the following enzymes: phenylalanine hydroxylase, tyrosinhydroxylase, tryptophanhydroxylase or NO-synthase.

Therewith the inventive chaperones are suited for therapy of conditions with elevated phenylalanine or reduced tyrosin, serotonin, or dopamine in body fluids, tissues or cells, in particular in conditions with reduced phenylalanine hydroxylase, tyrosinhydroxylase, tryptophanhydroxylase or NO-Synthase can be employed.

This aspect of the present invention concerns the use of at least one compound according to the following general formula as neurotransmitter or secondary messenger enhancer, in particular for catecholamine and/or serotonin and/or dopamine and/or nitric oxide (NO);

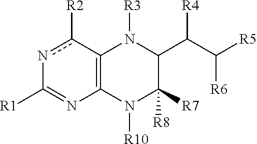

wherein R1 is selected from the group consisting of: H, OH, SH, F, Cl, Br, I, NH₂, N(CH₃)₂, N(C₂H₅)₂, N(C₃H₇)₂; NH-acyl, wherein the acyl residue contains 1 to 32 carbon atoms, in particular CH₃O, preferably 9 to 32, preferably 9 to 20 carbon atoms;
wherein R2 is selected from the group consisting of H, OH, SH, NH₂, F, Cl, Br, I, O, S;
wherein R3 is selected from the group consisting of: H, CH₃, C₂H₅;
wherein R4 and R6 are selected independently of each other from the group consisting of: H, OH, SH, NH₂, F, Cl, Br, I, acetyl, OX, wherein X is a C1 to C32 acyl residue, in particular a C9 to C32 acyl residue, preferably a C9 to C20 acyl residue;
wherein R5 is selected from the group consisting of: phenyl, CH₃, C₂H₅, C₃H₇, butyl, isobutyl, t-butyl;
wherein R7 and R8 are selected independent of each other from the group consisting of: H, OH, SH, NH₂, F, Cl, Br, I, CH₃, COOH, CHO, COOR9, wherein R9 CH₃, C₂H₅, C₃H₇, butyl;
wherein R10 is selected from the group consisting of: H, CH₃, C₂H₅, and -- represents an optional double bond; as well as their pharmaceutically acceptable salts.

Also as neurotransmitter or secondary messenger enhancer there is preferably selected a compound from the group consisting of: 5, 6, 7, 8-tetrahydrobiopterine, sapropterin, in particular the hydrochloride thereof, as well as the compound with the following structure:

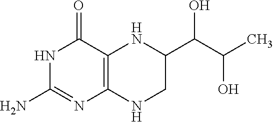 [SMILES: CC(O)C(O)C1CNc2nc(N)[nH]c(=O)c2N1]

(−)-(1′R,2'S,6R)-2-amino-6-(1′,2′-dihydroxypropyl)-5,6,7,8-tetrahydro-4(3H)-pteridinone,
   in particular their dihydrochlorides or sulfates and/or
2-N-stearoyl-1′,2′-di-O-acetyl-5,6,7,8-tetrahydrobiopterine; and/or
2-N-decanoyl-1′,2′-di-O-acetyl-5,6,7,8-tetrahydrobiopterine; and/or
2-N-palmitoyl-1′,2′-di-O-acetyl-5,6,7,8-tetrahydrobiopterine; and/or
2-N-linoleoyl-1′,2′-di-O-acetyl-5,6,7,8-tetrahydrobiopterine.

The present invention further concerns a composition, which contains at least one compound with the following general formula:

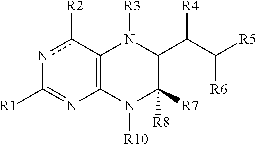

wherein R1 is selected from the group consisting of: H, OH, SH, F, Cl, Br, I, NH₂, N(CH₃)₂, N(C₂H₅)₂, N(C₃H₇)₂; NH-acyl, wherein the acyl residue contains 1 to 32 carbon atoms, in particular CH₃O, preferably 9 to 32, preferably 9 to 20 carbon atoms;
wherein R2 is selected from the group consisting of H, OH, SH, NH₂, F, Cl, Br, I, O, S;
wherein R3 is selected from the group consisting of: H, CH₃, C₂H₅;
wherein R4 and R6 are selected independent of each other from the group consisting of: H, OH, SH, NH₂, F, Cl, Br, I, acetyl, OX, wherein X is a C1 to C32 acyl residue, in particular a C9 to C32 acyl residue, preferably a C9 to C20 acyl residue;
wherein R5 is selected from the group consisting of: phenyl, CH₃, C₂H₅, C₃H₇, butyl, isobutyl, t-butyl;
wherein R7 and R8 are selected independent of each other from the group consisting of: H, OH, SH, NH₂, F, Cl, Br, I, CH₃, COOH, CHO, COOR9, wherein R9 CH₃, C₂H₅, C₃H₇, butyl;
wherein R10 is selected from the group consisting of: H, CH₃, C₂H₅, and -- represents an optional double bond; as well as their pharmaceutically acceptable salts; as well as at least one amino acid, which is selected from the group consisting of the essential amino acids: isoleucine, leucine, lysine, methionine, threonine, tryptophane, valine, histidine; as well as from the non-essential amino acids, in particular alanine, arginine, asparaginic acid, asparagine, cysteine, in particular acetylcysteine glutamine acid, glutamine, glycine, proline, serine as well as tyrosine.

One preferred composition is characterized thereby, that it contains the essential amino acids, selected from the group consisting of: isoleucine, leucine, lysine, methionine, threonine, tryptophane, valine, histidine and supplementally at least one of the amino acids alanine, arginine, asparaginic acid, asparagine, cysteine, in particular acetylcysteine, glutamic acid, glutamine, clycin, prolin, serine as well as tyrosin.

It is further preferred that the inventive composition contain carbohydrates, in particular glucose and/or vitamins.

Preferably the inventive composition can be formulated as a preparation to be administered orally or intravenously.

The preparation can be formulated in the form of a powder, tablet, capsule, pill, droplets or for topical application, in particular as a salve or cream; as well as a solution for intravenous administration.

Of course this type of preparation can be in the form of pharmaceutical compositions, in certain cases with conventional pharmaceutical galenic aids.

The inventive composition can however likewise be in the form of dietetic composition, in certain cases with consumable technology conventional aids, in particular emulsifiers, preferably lecitin or choline.

Beyond this it is preferred that the inventive composition contains additional minerals and/or electrolytes, which can be selected from: mineral salts; saline salts; sea salts; trace elements, in particular selenium, manganese, copper, zinc, molybdenum, iodine, chrome; alkali ions, in particular lithium, sodium, potassium; earth alkali ions, in particular magnesium, calcium; iron.

In the framework of a dietetic nutrient for patients with hyperphenylalaninemia the inventive composition can even supplementally contain phenylalanine, without the occurrence of the danger of a toxic accumulation of phenylalanine in the serum, cerebral spinal fluid and/or the brain.

Further it is preferred that the composition supplementally contain L-carnitine and/or myoinositole and/or choline.

Beyond this it can be useful when the inventive composition contains one of the anti-oxidants conventional in foodstuffs, in particular Vitamin C, whereby the oxidative decomposition of the tetrahydrobiopterine derivative can at least be substantially avoided and the storage stability of the composition be improved.

Beyond this, a composition with a compound is employed, wherein the compound is selected from the group consisting of: 5, 6, 7, 8-tetrahydrobiopterine, sapropterin, in particular the hydrochloride thereof, as well as the compound with the following structure:

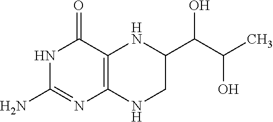 [SMILES: CC(O)C(O)C1CNc2nc(N)[nH]c(=O)c2N1]

(−)-(1′R,2′S,6R)-2-amino-6-(1′,2′-dihydroxypropyl)-5,6,7,8-tetrahydro-4(3H)-pteridinone,
   in particular their dihydrochlorides or sulfates and/or
2-N-stearoyl-1′,2′-di-O-acetyl-5,6,7,8-tetrahydrobiopterine; and/or
2-N-decanoyl-1′,2′-di-O-acetyl-5,6,7,8-tetrahydrobiopterine; and/or
2-N-palmitoyl-1′,2′-di-O-acetyl-5,6,7,8-tetrahydrobiopterine; and/or
2-N-linoleoyl-1′,2′-di-O-acetyl-5,6,7, 8-tetrahydrobiopterine.

The present invention derives particular significance in the manufacture of nutrient supplements, which are suitable for making possible in patients afflicted with amino acid metabolism disturbances a substantially normal diet despite their affliction.

In particular the present invention concerns the use of at least one compound with the following general formula:

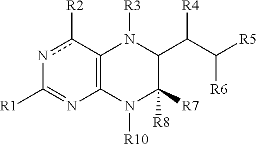

wherein R1 is selected from the group consisting of: H, OH, SH, F, Cl, Br, I, NH₂, N(CH₃)₂, N(C₂H₅)₂, N(C₃H₇)₂; NH-acyl, wherein the acyl residue contains 1 to 32 carbon atoms, in particular CH₃O, preferably 9 to 32, preferably 9 to 20 carbon atoms;
wherein R2 is selected from the group consisting of H, OH, SH, NH₂, F, Cl, Br, I, O, S;
wherein R3 is selected from the group consisting of: H, CH₃, C₂H₅;
wherein R4 and R6 are selected independent of each other from the group consisting of: H, OH, SH, NH₂, F, Cl, Br, I, acetyl, OX, wherein X is a C1 to C32 acyl residue, in particular a C9 to C32 acyl residue, preferably a C9 to C20 acyl residue;
wherein R5 is selected from the group consisting of: phenyl, CH₃, C₂H₅, C₃H₇, butyl, isobutyl, t-butyl;
wherein R7 and R8 are selected independent of each other from the group consisting of: H, OH, SH, NH₂, F, Cl, Br, I, CH₃, COOH, CHO, COOR9, wherein R9 CH₃, C₂H₅, C₃H₇, butyl;
wherein R10 is selected from the group consisting of: H, CH₃, C₂H₅, and -- represents an optional double bond; as well as their pharmaceutically acceptable salts, as nutrient supplements.

As nutrient supplement for the challenged patient group there is suited in particular one such compound, which is selected from the group consisting of:

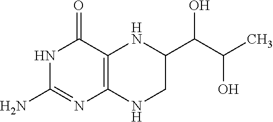 [SMILES: CC(O)C(O)C1CNc2nc(N)[nH]c(=O)c2N1]

(−)-(1′R,2'S,6R)-2-amino-6-(1′,2′-dihydroxypropyl)-5,6,7,8-tetrahydro-4(311)-pteridinone,
   in particular their dihydrochlorides or sulfates and/or
2-N-stearoyl-1′,2′-di-O-acetyl-5,6,7,8-tetrahydrobiopterine; and/or
2-N-dDecanoyl-1′,2′-di-O-acetyl-5,6,7,8-tetrahydrobiopterine; and/or
2-N-palmitoyl-1′,2′-di-O-acetyl-5,6,7,8-tetrahydrobiopterine; and/or
2-N-linoleoyl-1′,2′-di-O-acetyl-5,6,7,8-tetrahydrobiopterine.

The present invention finds exceptional significance in the manufacture of a special nutrient on the basis of essentially phenylalanine-free amino acid mixtures, with which in particular patients with hyperphenylalaninemia can optimally be nurtured.

This type of special nutrient contains preferably a compound with the following general formula:

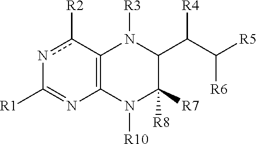

wherein R1 is selected from the group consisting of: H, OH, SH, F, Cl, Br, I, NH₂, N(CH₃)₂, N(C₂H₅)₂, N(C₃H₇)₂; NH-acyl, wherein the acyl residue contains 1 to 32 carbon atoms, in particular CH₃O, preferably 9 to 32, preferably 9 to 20 carbon atoms;
wherein R2 is selected from the group consisting of H, OH, SH, NH₂, F, Cl, Br, I, O, S;
wherein R3 is selected from the group consisting of: H, CH₃, C₂H₅;
wherein R4 and R6 are selected independent of each other from the group consisting of: H, OH, SH, NH₂, F, Cl, Br, I, acetyl, OX, wherein X is a C1 to C32 acyl residue, in particular a C9 to C32 acyl residue, preferably a C9 to C20 acyl residue;
wherein R5 is selected from the group consisting of: phenyl, CH₃, C₂H₅, C₃H₇, butyl, isobutyl, t-butyl;
wherein R7 and R8 are selected independent of each other from the group consisting of: H, OH, SH, NH₂, F, Cl, Br, I, CH₃, COOH, CHO, COOR9, wherein R9 CH₃, C₂H₅, C₃H₇, butyl;
wherein R10 is selected from the group consisting of: H, CH₃, C₂H₅, and -- represents an optional double bond; as well as their pharmaceutically acceptable salts.

As special nutrient for hyperphenylalaninamie patients, there is particularly suited one such composition which contains at least one compound which is selected from the group consisting of:

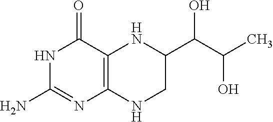 [SMILES: CC(O)C(O)C1CNc2nc(N)[nH]c(=O)c2N1]

(−)-(1′R,2′S,6R)-2-amino-6-(1′,2′-dihydroxypropyl)-5,6,7,8-tetrahydro-4(3H)-pteridinone,
   in particular there dihydrochlorides or sulfates and/or
2-N-stearoyl-1′,2′-di-O-acetyl-5,6,7,8-tetrahydrobiopterine; and/or
2-N-decanoyl-1′,2′-di-O-acetyl-5,6,7,8-tetrahydrobiopterine; and/or
2-N-palmitoyl-1′,2′-di-O-acetyl-5,6,7,8-tetrahydrobiopterine; and/or
2-N-linoleoyl-1′,2′-di-O-acetyl-5,6,7,8-tetrahydrobiopterine.

For ensuring the complete nutrient offerings it is preferred that the inventive special nutritional formulation supplementally contains carbohydrates, in particular glucose, maltodextrin, starch and/or fats, such as fish oil, in particular salmon oil, herring oil, mackerel oil or tuna fish oil.

It is particularly preferred that the special nutritional formulation is hypoallergenic and/or essentially glutenin/gluten free.

Since most amino acid metabolic disorders are genetically caused diseases, it is necessary to provide the patients with the correct nutrients from birth on. Thus it is of particular advantage, that the special diet according to the present invention can be formulated as infant formula, in particular as milk substitute both for infants as well as older children and adults.

One milk substitute for infants of this type comprises supplementally a fat component, wherein in particular approximately 90% are present in the form of triglycerides, 10% as mono and diglycerides.

For the light confectioning and for increasing the storage stability the special nutrient is available as powder, in particular as lyophilisate.

It is further preferred to supplement the inventive special nutrient with fatty acid supplements, in particular unsaturated fatty acids, preferably omega-3-fatty acids, in particular alphalinoleic acid, docosahexanoic acid, eicosapentaenic acid or omega-6 fatty acids, in particular arachidonic acid, linolic acid, linolenic acid or oleic acid.

It is further preferred that the special nutrient contain fish oil supplements, in particular from salmon, herring, mackerel or tuna fish oil.

Beyond this the special nutrient can include a fat component, which includes the vegetable oils, in particular safflower oil and/or soy oil and/or cocoa oil.

A further preferred embodiment of the special nutrient of the present invention can be developed in the form of a milk substitute on the basis of its character also as special nutrient for patients with an amino acid metabolic disturbance, in particular hyperphenylalaninamie, in particular a fruit milk mix drink or chocolate milk.

In the nourishment of patients with hyperphenylalaninamie the present invention has a particular excellent significance: by the accomplishment of the present invention by the inventor, it is for the first time possible to make available for such patients a phenylalanine-poor special nutrient, which by the supplementation of tetrahydrobiopterine-derivatives is suited for increasing the protein tolerance and the decomposition of phenylalanine.

According to the present invention one such phenylalanine poor special nutrient contains a protein poor base nutrient means, as well as at least one compound with the following general formula:

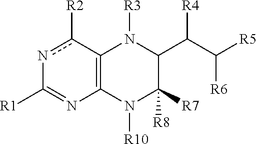

wherein R1 is selected from the group consisting of: H, OH, SH, F, Cl, Br, I, NH₂, N(CH₃)₂, N(C₂H₅)₂, N(C₃H₇)₂; NH-acyl, wherein the acyl residue contains 1 to 32 carbon atoms, in particular CH₃O, preferably 9 to 32, preferably 9 to 20 carbon atoms;
wherein R2 is selected from the group consisting of H, OH, SH, NH₂, F, Cl, Br, I, O, S;
wherein R3 is selected from the group consisting of: H, CH₃, C₂H₅;
wherein R4 and R6 are selected independent of each other from the group consisting of: H, OH, SH, NH₂, F, Cl, Br, I, acetyl, OX, wherein X is a C1 to C32 acyl residue, in particular a C9 to C32 acyl residue, preferably a C9 to C20 acyl residue;
wherein R5 is selected from the group consisting of: phenyl, CH₃, C₂H₅, C₃H₇, butyl, isobutyl, t-butyl;
wherein R7 and R8 are selected independent of each other from the group consisting of: H, OH, SH, NH₂, F, Cl, Br, I, CH₃, COOH, CHO, COOR9, wherein R9 CH₃, C₂H₅, C₃H₇, butyl;
wherein R10 is selected from the group consisting of: H, CH₃, C₂H₅, and -- represents an optional double bond; as well as their nutritionally acceptable salts.

For the inventive phenylalanine poor special nutrient it is likewise preferred to employ a compound which is selected from the group consisting of:

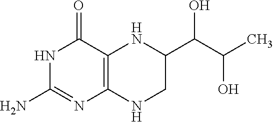 [SMILES: CC(O)C(O)C1CNc2nc(N)[nH]c(=O)c2N1]

(−)-(1′R,2′S,6R)-2-amino-6-(1′,2′-dihydroxypropyl)-5,6,7,8-tetrahydro-4(3H)-pteridinone,
   in particular their dihydrochlorides or sulfates and/or
2-N-stearoyl-1′,2′-di-O-acetyl-5,6,7,8-tetrahydrobiopterine; and/or
2-N-decanoyl-1′,2′-di-O-acetyl-5,6,7,8-tetrahydrobiopterine; and/or
2-N-palmitoyl-1′,2′-di-O-acetyl-5,6,7,8-tetrahydrobiopterine; and/or
2-N-linoleoyl-1′,2′-di-O-acetyl-5,6,7,8-tetrahydrobiopterine.

It is possible and preferred to formulate the phenylalanine poor special nutrient as: finished dishes; dough products, in particular noodles; baked products, in particular bread, cake, biscuits; sweets, in particular chocolate, candy, ice cream; drinks, in particular artificial milk, in the form of milk mix drinks, in particular as fruit milk mix drink or chocolate, as well as beer.

Therewith hyperphenylalaninamie patients can for the first time partake of significantly higher amounts of traditional fare without risk of danger on the basis of their amino acid metabolic disorder—and without having to be exclusively limited to the bad-tasting products which are the state of the art.

As a consequence of the rapid onset of the effect of tetrahydrobiopterine derivatives it is supplementally possible within the framework of the present invention to provide a diagnostic for recognizing tetrahydrobiopterine sensitive diseases of amino acid metabolism, which contains at least one compound with the following general formula:

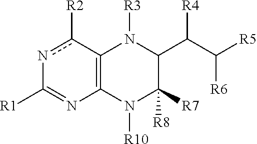

wherein R1 is selected from the group consisting of: H, OH, SH, F, Cl, Br, I, NH₂, N(CH₃)₂; N(C₂H₅)₂, N(C₃H₇)₂; NH-acyl, wherein the acyl residue contains 1 to 32 carbon atoms, in particular CH₃O, preferably 9 to 32, preferably 9 to 20 carbon atoms;
wherein R2 is selected from the group consisting of H, OH, SH, NH₂, F, Cl, Br, I, O, S;
wherein R3 is selected from the group consisting of: H, CH₃, C₂H₅;
wherein R4 and R6 are selected independent of each other from the group consisting of: H, OH, SH, NH₂, F, Cl, Br, I, acetyl, OX, wherein X is a C1 to C32 acyl residue, in particular a C9 to C32 acyl residue, preferably a C9 to C20 acyl residue;
wherein R5 is selected from the group consisting of: phenyl, CH₃, C₂H₅, C₃H₇, butyl, isobutyl, t-butyl;
wherein R7 and R8 are selected independent of each other from the group consisting of: H, OH, SH, NH₂, F, Cl, Br, I, CH₃, COOH, CHO, COOR9, wherein R9 CH₃, C₂H₅, C₃H₇, butyl;
wherein R10 is selected from the group consisting of: H, CH₃, C₂H₅, and -- represents an optional double bond;
in particular 5, 6, 7, 8-tetrahydrobiopterine; as well as their pharmaceutically acceptable salts.

In summary it can be concluded that it is possible for the first time with the compounds described within the framework of the invention to treat certain genetically caused amino acid metabolism diseases without medication, so that the patient exhibits an improvement in protein tolerance as well as a substantial normalization of their disturbed enzyme activity as well as the concentrations of the concerned amino acids and/or their metabolic products in body fluids and body cells.

Further, the present invention proposes compositions of nutrient supplement means and special nutrients which at the same time contain the compounds described in the invention for improvement of protein tolerance and for the decomposition of phenylalanine. Thereby it is possible for the first time to feed patients with amino acid metabolic disturbances practically normally, that is, with quasi all taste and composition nuances.

Besides the above already repeatedly mentioned compounds, the following compounds can however also find application as preferred embodiments for the various claim categories: The various individual components as well as their various enantiomers, which result from the respective disclosed substituents R1 through R10 and X from the shown general formula as well as various subcombinations thereof.

In particular the following subcombinations of compounds are a component of the present disclosure:

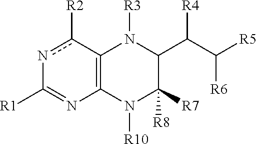

wherein R1 is selected from the group consisting of: H, OH, SH; and/or
wherein R1 is selected from the group consisting of: F, Cl, Br, I; and/or
wherein R1 is selected from the group consisting of: NH₂, N(CH₃)₂, N(C₂H₅)₂, N(C₃H₇)₂; and/or
wherein R1 is NH-acyl, wherein the acyl residue contains 1 to 32 carbon atoms, in particular CH₃O, preferably 9 to 32, preferably 9 to 20 carbon atoms; and/or
wherein R2 is selected from the group consisting of: H, OH, SH; and/or
wherein R2 is selected from the group consisting of: NH₂, F, Cl, Br, I, O, S; and/or
wherein R3 is selected from the group consisting of: H, CH₃, C₂H₅; and/or
wherein R4 and R6 independent of each other are selected from the group consisting of: H, OH, SH, NH₂, and/or
wherein R4 and R6 independent of each other are selected from the group consisting of: F, Cl, Br, I; and/or
wherein R4 and R6 independent of each other are acetyl; and/or
wherein R4 and R6 independent of each other are selected from the group consisting of: OX, wherein X is a C1 to C32 acyl residue, in particular a C9 to C32 acyl residue, preferably a C9 to C20 acyl residue; and/or
wherein R5 is selected from the group consisting of: CH₃, C₂H₅, C₃H₇, butyl, isobutyl, t-butyl; and/or
wherein R5 is phenyl; and/or
wherein R7 and R8 independent of each other are selected from the group consisting of: H, OH, SH, NH₂, F, Cl, Br, I, CH₃, COOH, CHO; and/or
wherein R7 and R8 independent of each other are selected from the group consisting of: COOR9, wherein R9 is CH₃, C₂H₅, C₃H₇, butyl; and/or
wherein R10 is selected from the group consisting of: H, CH₃, C₂H₅, and -- represents an optional double bond.

It has further been discovered that lipophilic tetrahydrobiopterine derivatives, as described for example in EP 0 164 964 A1, are particularly suited, in order on the one hand to elevate this serum residence time in comparison to tetrahydrobiopterine from approximately 8 hours to greater than 18 hours. On the other hand this type of lipophilic tetrahydrobiopterine derivative is particularly suited in order to produce special nutrients and nutrient supplements since they dissolve readily in fat-containing mixtures, for example, artificial milk compositions.

Further the advantage of the lipophilic compounds is in their reduced oxidation sensitivity.

This type of lipophilic compounds are in particularly those, in which

R1 in the above general formula is a NH-acyl, wherein the acyl residue is in particular 9 to 32, preferably 9 to 20 carbon atoms, contains; and/or

R4 and R6 independent of each other are selected from the group consisting of: OX, wherein X is in particular a C9 to C32 acyl residue, preferably a C9 to C20 acyl residue, wherein the substituents R2, R3, R5, R7, R8, R9, R10 can be selected as disclosed in the framework of the present invention.

Preferably the following lipophilic tetrahydrobiopterine derivatives can be employed for the purposes of the present invention:
2-N-stearoyl-1′,2′-di-O-acetyl-5,6,7,8-tetrahydrobiopterine; and/or
2-N-decanoyl-1′,2′-di-O-acetyl-5,6,7,8-tetrahydrobiopterine; and/or
2-N-palmitoyl-1′,2′-di-O-acetyl-5,6,7,8-tetrahydrobiopterine; and/or
2-N-linoleoyl-1′,2′-di-O-acetyl-5,6,7,8-tetrahydrobiopterine.

Tetrahydrobiopterine is at this time commercially available, for example as sapropterinhydrochloride which is available under the name BIOPTEN® from the company Suntory and which is employed for therapy of genetically dependent tetrahydrobiopterine synthesis efficiencies or disturbances.

Beyond this, tetrahydrobiopterine and its derivatives can be synthetically produced. For example, for this the teaching of EP 0 164 964 A1 is mentioned therefore, which among other things describes the production of a series of acylated tetrahydrobiopterine derivatives. Further, U.S. Pat. No. 4,665,182 describes the organic chemical synthesis of biopterine derivatives.

Accordingly, the manufacture of the employed compounds is not a problem to the person of ordinary skill.

Further advantages and characteristics can be seen on the basis of the description of illustrative embodiments as well on the basis of the figures. There is shown in

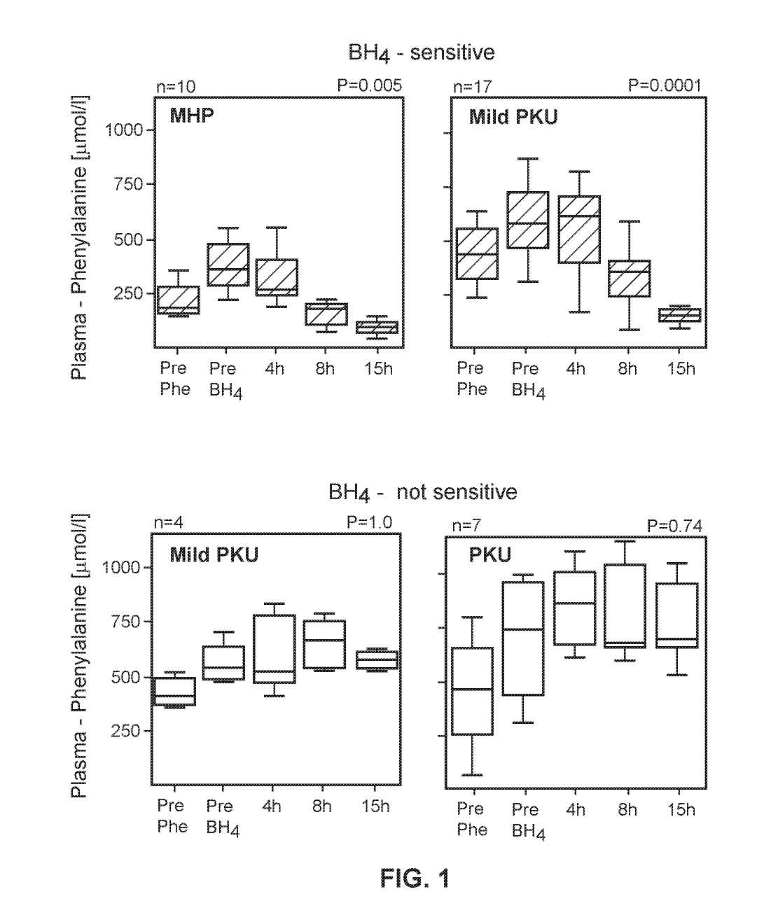

FIG. 1 the phenylalanine concentration in blood prior to provocation with phenylalanine as well as prior to and following administration of tetrahydrobiopterine in mild hyperphenylalaninamie, mild phenylketonurea, mild phenylketonurea not responsive to tetrahydrobiopterine as well as classical phenylketonurea;

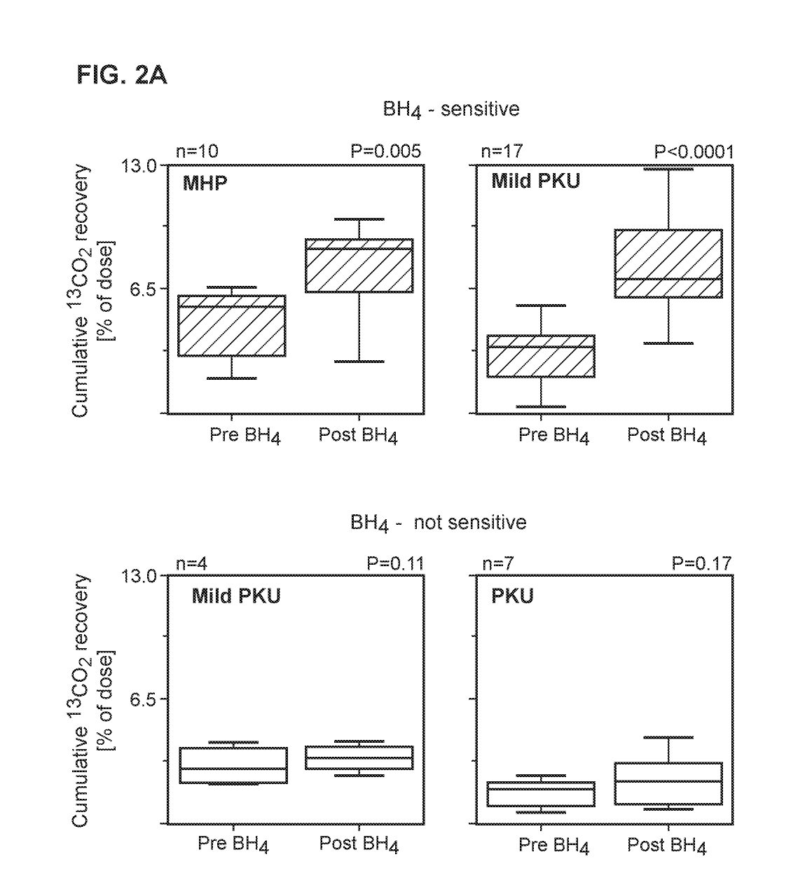

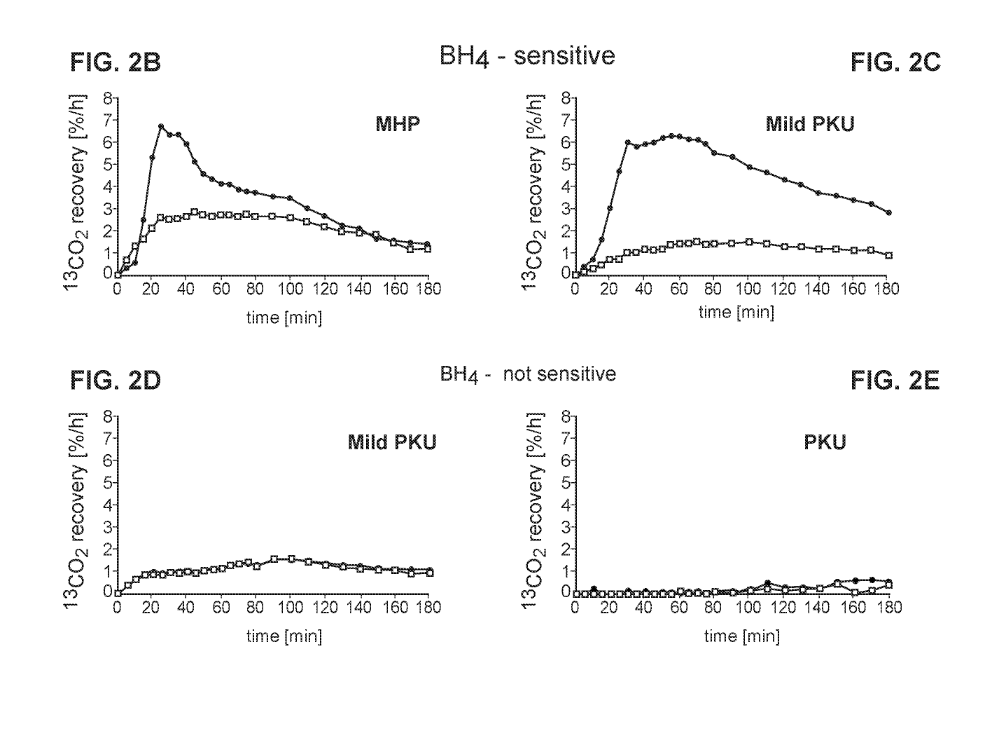

FIG. 2 the effect of short time treatment with tetrahydrobiopterine on phenylalanine oxidation;

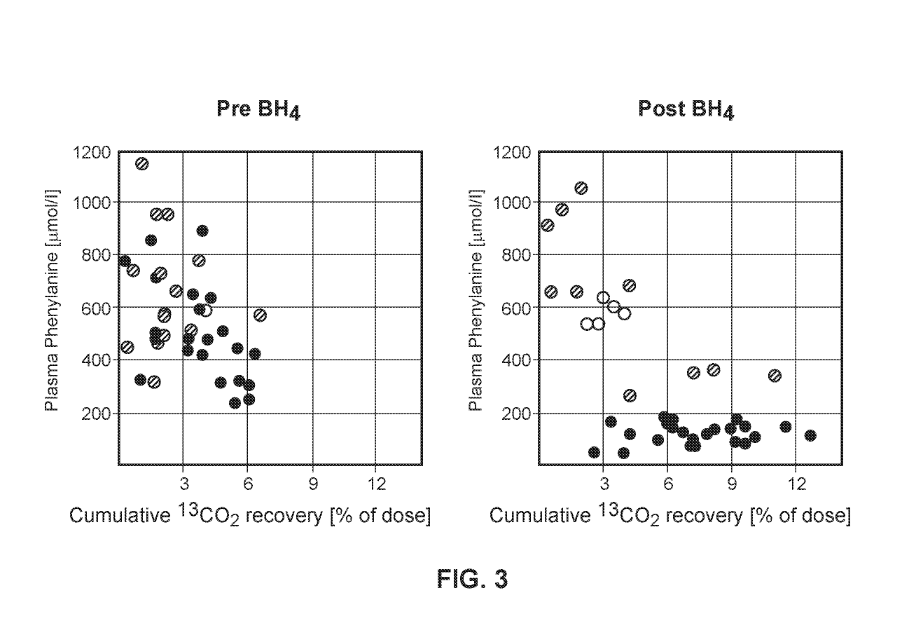

FIG. 3 a relation between the cumulative persistence of C-marked CO₂ during the administration of C-marked phenylalanine and the phenylalanine-blood concentration prior to and subsequent to administration of tetrahydrobiopterine;

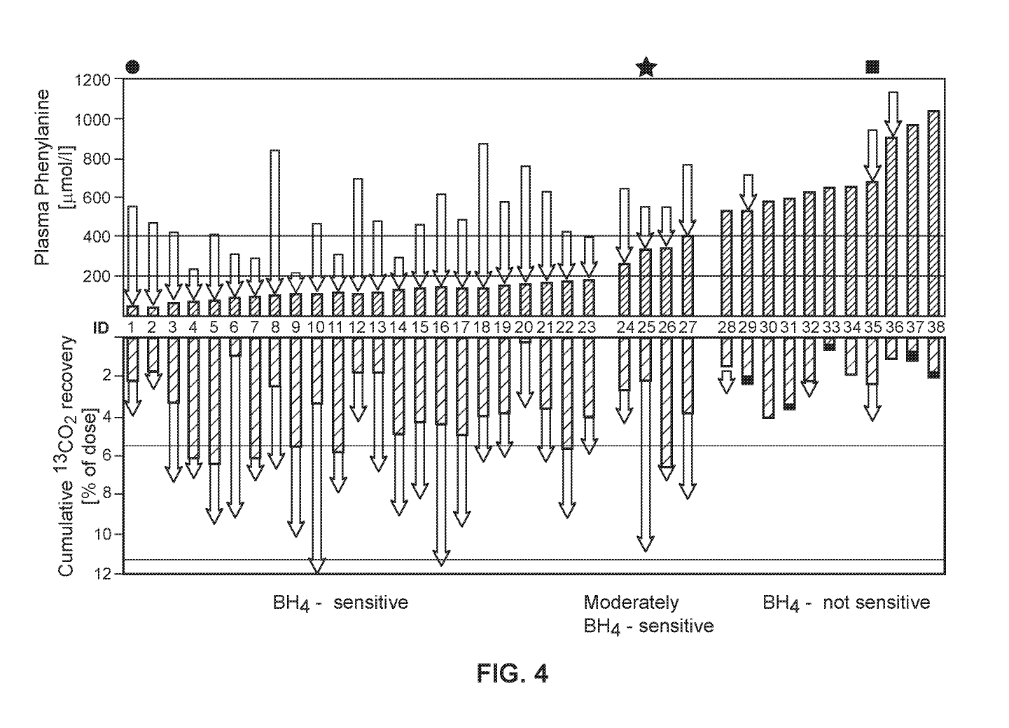

FIG. 4 the effect of tetrahydrobiopterine on the peripheral phenylalanine-clearance and oxidation rate in patients with hyperphenylalaninamie; and

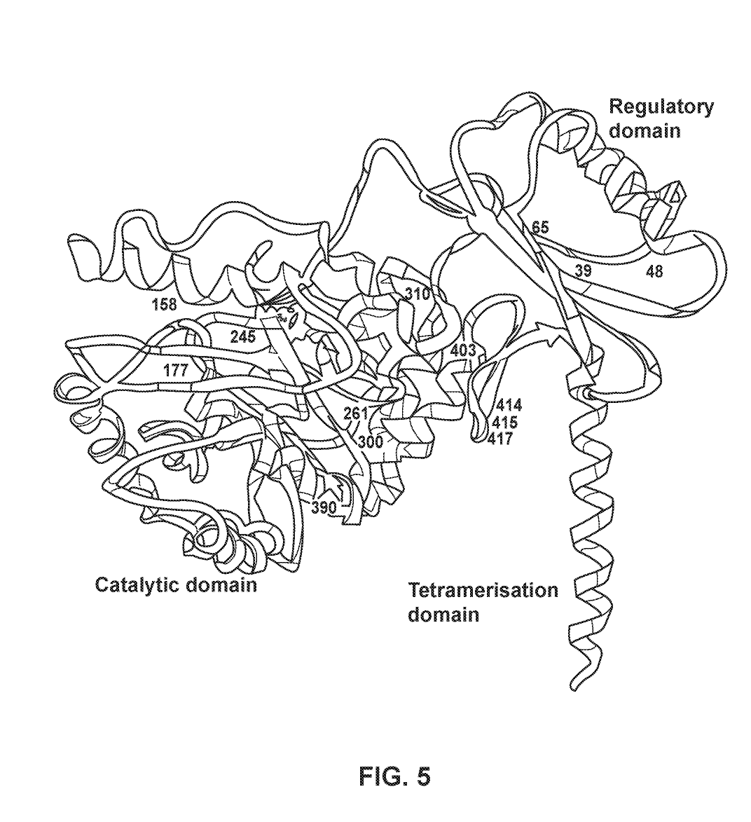

FIG. 5 the structural localization of phenylalanine hydroxylase missense-mutations.

Table 1 the correlation of the genotypes to clinical phenotypes.

### EXAMPLE

### Methodology

In order to research the therapeutic effectiveness of tetrahydrobiopterine, one carries out a combined phenylalanine tetrahydrobiopterine stress test for diagnostic and analyzes the effect in vivo by means of determining the [¹³C] phenylalanine oxidation rate and 38 persons with a deficiency in phenylalanine hydroxylase prior to and subsequent to the administration of tetrahydrobiopterine derivatives. The response to tetrahydrobiopterine was associated with certain genotypes, and we localized mutations on the basis of the structural models of the phenylalanine hydroxylase monomer and the therefrom derived protein misfolding.

### Results

In 27 of the 31 patients (87%) with mild hyperphenylalaninamie (n=10) or mild phenylketonurea (n=21) the tetrahydrobiopterine significantly decreased the phenylalanine content in the blood and elevated/improved the phenylalanine oxidation. On the other hand, none of these seven patients with classical phenylketonurea (n=7) satisfy the criteria of a strong response to tetrahydrobiopterine, as defined in the study. In individual patients with classical phenylketonurea small effects were however exhibited. A long time therapy with tetrahydrobiopterine, which was carried out in five children, elevated the daily phenylalanine tolerance significantly from 8.7±8.6 mg/kg body weight (range 8.8-30) to 61.4±27.9 mg/kg body weight (range 17.9-90) with medication-free treatment (P=0.0043) and therewith made it possible for them to discontinue their special diet. Seven mutations of the phenylalanine hydroxylase gene (P314S, Y417H, V177M, V245A, A300S, E290G and IVS4-5C→G) and the therefrom resulting structural anomaly and misfolding of the enzyme were classified as the highest probability of the cause in association with the response of the tetrahydrobiopterine and six mutations (A403V, F39L, D415N, S310Y, R158Q and I65T) were classified as possibly having some association. Four mutations (Y414C, L48S, R261Q and 165V) showed no consistent unity (of reaction) with this phenotype. With the mutations associated with a response to tetrahydrobiopterine, these were above all localized in the catalytic area of the protein and were not directly involved in the cofactor formation.

### Resulting Conclusions:

A response to tetrahydrobiopterine derivative—characterized by improvement in protein tolerance, substantial normalization of disrupted phenylalanine hydroxylase activity as well as reduction of elevated phenylalanine concentration—occurred frequently in patients with a mild phenotype of hyperphenylalaninamie. The response cannot be reliably predicted on the basis of the genotype, which applied above all in the composite double heterozygote genotype. The medication-free treatment of with tetrahydrobiopterines and/or supplementation of the compounds to nutrients was able to relieve or free many patients from their burdensome phenylalanine-poor diet and thereby facilitate their nourishment or diet.

After filing of the present patent application the data reflecting the invention will be published in scientific credible form and documented: New England Journal of Medicine, 2002, 347 (26), 2122-2132 (26.12.02).

### Introduction

Hyperphenylalaninamie, a broad spread inheritable medical condition, was one of the first genetic afflictions which could be treated. In most cases hyperphenylalaninamie resulted from a lack of phenylalanine hydroxylase (EC1.14.16.1), where about by mutations on the phenylalanine hydroxylase gene. The therewith associated phenotypes range in their degree of seriousness from classical phenylketonurea (MIM261600) through mild phenylketonurea and mild hyperphenylalaninamie. At least half of the concerned patients suffered from one of the milder clinical phenotypes. Both patients which suffer from classical phenylketonurea as well as patients which suffer from a mild phenylketonurea must partake over their life of a protein-poor diet, in order to avoid neurological consequential symptoms and to insure a normal cognitive development. In association with a very strict special diet there exists the risk of nutritionally dependent deficiency symptoms, at least it represents a burden for the patients and their families. Only patients which suffer from a mild hyperphenylalaninamie require in certain cases no treatment. The search for alternative treatment methods without changing the nutritional diet is actively ongoing.

For approximately 50 genetic origin illnesses in humans the treatment can be stimulated by a high dose of a cofactor of the enzyme activity. Tetrahydrobiopterine is a natural cofactor of aromatic amino acid hydroxylases and nitrogen oxide synthase. The substitution of this cofactor component is an established treatment method in rare cases of hyperphenylalaninamie, which is caused by inherited defects in the tetrahydrobiopterine biosynthesis. More than 98% of the patients with hyperphenylalaninamie exhibit however mutations on the phenylalanine hydroxylase gene and they more likely have an elevated than a reduced plasma concentration of biopterine, which can be traced back to activity of the guanosine triphosphate cyclohydroxylase I-feedback regulation protein. A possible therapeutic effect of the tetrahydrobiopterine in patients with a lack of phenylalanine hydroxylase was, for this reason, not considered until now.

In recent times it was demonstrated that individual patients with mutations of the phenylalanine hydroxylase gene exhibited low concentrations of phenylalanine in the blood, after they were supplied with tetrahydrobiopterine for diagnostic purposes. It is however known, that peripheral phenylalanine values of various genetic location and mutating or changing factors are regulated, and there is no proof, that the positive effect of tetrahydrobiopterine occurs on the level of the phenylalanine hydroxylation.

In this study which was carried out on the basis of patients selected at random, the following questions were considered:

(1) How broadly is the response to tetrahydrobiopterine distributed? (2) Does tetrahydrobiopterine reestablish the phenylalanine oxidation capability? (3) Is the response to tetrahydrobiopterine linked with certain genotypes and are the therewith associated mutations located on specific locations on the protein structure? (4) Does the protein tolerance improve with long term treatment?

### Process

### Patients

We obtained the written consent of the families of 38 children, which suffered from various subset forms of hyperphenylalaninamie. The classification occurred depending upon the plasma phenylalanine concentration prior to treatment: less than 600 μmol/l, mild hyperphenylalaninamie, n=10, age 15 days through 10 years; 600-1200 μmol/l, mild, n=21, age 8 days through 17 years; greater than 1200 μmol, classical phenylketonurea, n=7, age 1 day through 9 years. A defect in this tetrahydrobiopterine biosynthesis or in the recycling of the tetrahydrobiopterine was ruled out by an analysis of the pterine value in urine and the dihydropteridine-reductase activity in erythrocytes. We examined seven patients during the newborn period and 31 as they were already older. Afflicted siblings (n=5) were likewise included in the study, since it is known that non-genetic factors influence the phenylalanine homeostasis.

### Combined Phenylalanine and Tetrahydrobiopterine Exposure or Stress Test.

The uptake of phenylalanine was accomplished in that the patients were allowed to take a meal with 100 mg phenylalanine per kilogram body weight. One hour after the end of the meal the patients took 20 mg tetrahydrobiopterine per kilogram (Schircks Laboratories, Jona, Switzerland). The phenylalanine concentration in blood was determined by an electro spray ionization tandem mass spectroscopy—prior to the uptake of phenylalanine and prior to and subsequent to (at 4, 8 and 15 hours) provocation or exposure to tetrahydrobiopterine. During the test phase the newborns were fed with mothers milk, while the older children received a standardized protein supply (10 mg phenylalanine per kg) between six and eight hours after the exposure to tetrahydrobiopterine.

### In Vivo Analysis of L-Phenylalanine Oxidation

The tests were carried out after a four hour fast in small children and an overnight fast in older children. Overall 6 mg L-[1⁻¹³C] phenylalanine (Euriostop, Paris, France) per kilogram body weight were taken in orally. The tracer was dissolved in a 25% dextrose solution (2 mg per milliliter). Subsequently breath samples were taken over a period of 180 minutes and stored in air-free glass pipes until analysis by means of isotope mass spectroscopy (deltaS, Thermoquest, Bremen). The recapture of carbon-13 in the breath samples was calculated, as described by Treacy et al, wherein a total carbon dioxide of 300 mmol per hour×cubic meter of body surface was assumed. The ¹³CO₂-production was represented as a cumulative percentage rate of the calculated dose against time. The validity of the results in the newborn could have been influenced by the nutrition or the fact that the collection of the breath sample is more difficult with them than with older children. The base line percent rate of ¹³C, measured at time point 0 did not differ significantly however in the newborns and the older children. The values were considered to be less than detectable, when the signal intensity of the atom percent—excess at point and time t, obtained by subtraction of the average base value, did not allow any sufficient differentiation from atmospheric ¹³CO₂. In cross section, less than one (older children) and less than two (newborns) of 27 sequential ¹³CO₂ measurements, which were obtained during the 180 minutes of an individual test, were not capable of interpretation. This had an indiscernible influence upon the final evaluation period.

### Analysis of the Mutations

DNA was extracted from the leucocytes according to a standard process. 13 genome fragments, which contained the entire coded sequence, as well as the exon flanking interon sequence of the phenylalanine hydroxylase gene were amplified by polymerized chain reaction (PCR), followed by direct sequencing.

### Structure-Based Localization of Phenylalanine Hydroxylase Gene Mutations

A total length model of the tetrahydrobiopterine bound phenylalanine hydroxylase was produced from the crystal structures of various truncated forms, in that the catalytic areas were superimposed by means of SWISS-MODEL/Swiss-Pdb viewer provided tools.

### End Result

### Effective Tetrahydrobiopterine on the Phenylalanine Concentration in Blood and the Phenylalanine Oxidation Rates

The patients were classified as reacting to tetrahydrobiopterine if the phenylalanine concentration in the blood 15 hours after the exposure to tetrahydrobiopterine sank by at least 30% in comparison to the value prior to the intake of tetrahydrobiopterine. A response to tetrahydrobiopterine was observed in all ten patients with a mild phenylalaninamie and in 17 of 21 patients with a mild phenylketonurea. Only four patients with a mild phenylketonurea and all seven patients with a classic phenylketonurea did not satisfy the criteria as responding to tetrahydrobiopterine (FIG. 1). In the patients the phenylalanine concentration rapidly sank, similar to as was observed in patients with a tetrahydrobiopterine synthesis defect, while others only slowly reacted and achieved the lowest phenylalanine concentration only 15 hours after the cofactor administration (data not shown).

Patients with various clinical stages of illness achieved basile cumulative ¹³CO₂ recapture rates, which respectively reflected their individual rest phenylalanine oxidation capacity (classic phenylketonurea, average value 1.4%; mild phenylketonurea, 3.1%; mild hyperphenylalaninamie, 5.6%; the healthy comparison group 9.0%). After the treatment with tetrahydrobiopterine (10 mg/kg body weight, 24 hours) the total ¹³CO₂ recapture rose significantly in the same patients, which had responded to the stress test. The rise was more clearly pronounced in patients with a mild phenylketonurea than in patients with a mild hyperphenylalaninamie (FIG. 2A). It is remarkable, that 8 of 11 patients which did not respond exhibited a mild rise in phenylalanine oxidation after short time therapy with tetrahydrobiopterine, at which time in three of these patients simultaneously also the phenylalanine content in blood was influenced. This is associated therewith that with longer therapies, also in the cases of hyperphenylalaninamie derivative, improvement by tetrahydrobiopterine could be achieved. The time curve of the fractionated ¹³CO₂ formation shows clear deviation from normal oxidation phenotype (FIGS. 2B, C, D and E). After factoring in cofactor the curve in patients, which responded to tetrahydrobiopterine, dropped to the normal value (FIGS. 2B and C), at which time the patients, which did not respond to tetrahydrobiopterine, remained unchanged.

Prior to the treatment with tetrahydrobiopterine patients exhibited phenylalanine concentrations in blood of greater than 200 μmol, and a cumulative ¹³CO₂ recapture lie below 7% with a notable crossover or overlap of the values of the patients which responded to and the patients which did not respond. After the administration of tetrahydrobiopterine two non-overlapping clusters formed in the two patient groups. Among the tetrahydrobiopterine sensitive patients there were four children, which exhibited a moderate response to tetrahydrobiopterine (FIG. 3).

A considerable inter-individual variability could be observed: the exposure to tetrahydrobiopterine reduced the phenylalanine concentration from 37 to 92%, when one compared the blood values prior to and 15 hours after administration of tetrahydrobiopterine. In 23 of the 27 patients reacting to tetrahydrobiopterine the phenylalanine concentration in the blood fell back to values of less than 200 μmol/l, at which time four patients achieved values between 200 and 400 μmol/l. In patients which did not react, the concentration of phenylalanine after the exposure always exceeded 400 μmol/l. Tetrahydrobiopterine elevated the ¹³C-phenylalanine oxidation rate by 10 to 91% and 22 of the 27 persons reacting to tetrahydrobiopterine achieved oxidation rates in a normal level. In the remaining five patients an improvement could be observed, a normal level was however not achieved. Although in general consistent, there were in many patients significant lack of unity of the tetrahydrobiopterine effect at the two analyzed end points (examples indicated in FIG. 4). In a patient with classic phenylketonurea there occurred a slight increase in the phenylalanine concentration in blood, as well as an improvement of the phenylalanine oxidation rate, however the patient did not satisfy the criteria of the strong response to tetrahydrobiopterine (FIG. 4).

Long Time Treatment with Tetrahydrobiopterine

The families with five children aged from 4 to 14 years with mild phenylketonurea consented to a therapeutic test, in which the phenylalanine poor diet was replaced by an oral administration of tetrahydrobiopterine in daily doses between 7.1 and 10.7 mg/kg body weight. The treatment lasted 207±51.3 days (average±SD; length 166-263). The cofactor treatment lead to an increase in the daily phenylalanine tolerance of 8.7±8.6 mg/kg body weight (length 8.8-30) previously at 61.4±27.9 mg/kg body weight(length 17.9-90) with treatment (P=0.0043) with low effect on the phenylalanine concentration in blood (during the dietetic treatment, 366±120 μmol/l; during the pure cofactor treatment, 378±173 μmol/l).

### Identification and Structure Based Localization of Phenylalanine Hydroxylase Gene Mutations

In 37 of 38 patients respectively two mutant alleles (Table 1) were identified. We classified 7 mutations (P314S, Y417H, V177M, V245A, A300S, E390G, IVS4-5C>G) as most probable responsible for the response or the reaction to tetrahydrobiopterine, since they either are shown in homozygote or functional hemizygot form. Six further mutations are possible, on the basis of a significant in vitro residual enzyme activity (A403V, F39L, D415N, R158Q, I65T) as already described above, or on the basis of a known heavy mutation on the second allele (S310Y) in combination with the response to tetrahydrobiopterine. Four mutations (Y414C, L48S, R261Q, I65V) showed a non-uniform association with the response to tetrahydrobiopterine. 8 of 12 missense-mutations, which are in association with the response to tetrahydrobiopterine, are located on the catalytic domain, in comparison to which two of the regulator domain and two on the tetramerisation domain. None of them had any effect on residues of the active center or on amino acids which direct directly with the cofactor (FIG. 5).

### Discussion

We show on multiple lines of proof, in order to make clear, that the metabolic phenotype of the lack of phenylalanine hydroxylase can be significantly modified by pharmacological doses of tetrahydrobiopterine or derivatives thereof. First, the intake of tetrahydrobiopterine lead in most patients with a phenylalanine hydroxylase rest enzyme activity to normal or approaching normal phenylalanine concentrations in blood, which suggests that the responsiveness to tetrahydrobiopterine in patients, which phenotypically exhibit only mild symptoms, is broad-based. Second, tetrahydrobiopterine elevated the remaining phenylalanine oxidation capability in these patient groups. Third, long term treatment with tetrahydrobiopterine lead to a significant improvement in the protein tolerance and dispensing with the necessity of a limited diet therapy.

We show that the in vitro phenylalanine oxidation test makes possible a classification of patients with hyperphenylalaninamie into various classes of different degrees of seriousness. These results correspond with the data regarding the ability of the process to measure phenylalanine hydroxylase-gene-dose. On the basis of the multi factor nature of the hyperphenylalaninamie the phenylalanine oxidation speed in the total body is not a simple equivalent to the phenylalanine hydroxylase activity. The decline of the phenylalanine content in blood was accompanied by an improvement in the in vivo phenylalanine oxidation capacity in all patients, which responded to tetrahydrobiopterine. All things considered, these observations correspond with the hypothesis that the malfunction of the enzyme and the interfered-with phenylalanine hydroxylase activity can be improved by tetrahydrobiopterine. The magnitude of the improvement in phenylalanine decomposition corresponds not always with the improvement in the phenylalanine, a not unexpected result for a genetic determined enzyme deficiency in general and for the deficiency in phenylalanine hydroxylase in particular. We observed slow and rapid reactions, likewise the variations in time sequence and in the relative amount of the ¹³CO₂ formation, which indicates, that tetrahydrobiopterine brings about its effects by various paths of action and—depending upon the degree of the protein malfolding—with various efficiency. Besides the proposal that a high dosed tetrahydrobiopterine treatment could compensate for a reduced affinity of the defective phenylalanine hydroxylase with respect to tetrahydrobiopterine, further manners of action must be taken into consideration.

A treatment with tetrahydrobiopterine could supplementally drive or highly regulate the phenylalanine hydroxylase gene expression, stabilize phenylalanine hydroxylase mRNA, facilitate the functional phenylalanine hydroxylase tetramer formation or protect an incorrectly folded enzyme protein from a proteolytic digestion.

Predictions regarding the phenotype on the basis of the genotype could be difficult in the case of complex diseases caused by multi-factor genetics, such as by hyperphenylalaninemia. In the group of the patients which responded to tetrahydrobiopterine, we identified primarily “mild” genotypes, in comparison to which the genotypes of the patients which did not respond were primarily “heavy”. The experimental suggestion towards the association of various mutations with the response to tetrahydrobiopterine are of varying consistency and predictions on the basis of genotype are thus above all difficult in the present double heterozygote. It is known, that the Y414C mutation occurs in more than one clinical phenotype. We identified these mutations in a functional hemizygotic stage in two patients with identical genotypes however different reactions to tetrahydrobiopterine. These observations could be explained thereby, that the influences of multiple modifying gene locations in hyperphenylalaninamie have different effects. In homozygotic condition, which allows one to conclude a homopolymer tetramer formation, it was determined, that the Y414C as well as the L48S mutations bring about a response to tetrahydrobiopterine. In the functional hemizygote condition we observe these mutations however in individuals with classical phenylketonurea, which do not react to tetrahydrobiopterine. In these conditions the heteropolymerization could inhibit the formation of functional tetramers.

Our data confirm the assumption, that most of the missense mutations associated with the response to tetrahydrobiopterine lie in the catalytic domains of the protein, however do not concern in the rest of the active center and also are not involved directly in the co-factor formation. These mutations could have an effect on the interaction between the domains in a monomer or influence rests of the contact surface of the dimer or tetramer and therewith lead to a misfolding of the protein and reduced enzyme activity. Tetrahydrobiopterers thus serve as a chemical chaperone and prevent this.

Previously in vitro expression analysis were employed in order to predict the functional influence of the phenylalanine hydroxylase gene mutations in vivo. An over-estimation of the phenylalanine hydroxylase activities in vitro in comparison to those in vivo could be observed thereby. This could be explained by the fact, that the in vitro expression analysis until now was carried out exclusively in the presence of high concentrations of natural or synthetic co-factors, which made more difficult a genotype-phenotype correlation. Revised experimental protocols should encompass a series of various tetrahydrobiopterine concentrations, in order to be able to evaluate the intrinsic degree of seriousness of the mutations.

Since one could not conclude from the pre-therapeutic plasma phenylalanine concentrations whether and how response is made to tetrahydrobiopterine, a new clinical classification system would be advisable: (1) Hyperphenylalaninamie, which is not responsive to tetrahydrobiopterine, (2) Hyperphenylalaninamie, which is responsive to tetrahydrobiopterine, including (a) a deficiency of phenylalanine hydroxylase responding to tetrahydrobiopterine and (b) interference in the tetrahydrobiopterine biosynthesis pathway. A phenylalanine tetrahydrobiopterine stress test or exposure test with an extended observation phase (≧15 hours) can reliably distinguish between patients which responded and patients which did not respond and should be carried out for all persons who suffer from a hyperphenylalaninamie in order to positively identify patients which could profit from a tetrahydrobiopterine treatment. Our study, which was restricted to a short time interval, does not exclude the possibility of unearthing underestimated effects even in individual patients with classical phenylketonurea observable only after a longer treatment.

Our results show that a long time therapy with tetrahydrobiopterine leads to an elevated phenylalanine tolerance. A co-factor treatment, in place of the burdensome special diet, is appropriate for many patients and one could expect that the treatment with tetrahydrobiopterine derivatives would lead to a substantial improvement in quality of life. In particular the supplementation of these compounds to consumables should substantially simplify the design of the otherwise very difficult diet. A tetrahydrobiopterine treatment could likewise be helpful in maternal phenylketonurea, since the strict metabolic adjustment during the pregnancy is very difficult, however very important, in order to avoid grave negative effects in the newborn. How reliable or side effect free the intake of tetrahydrobiopterine during pregnancy is was however not determined. Worldwide a total of more than 350 patients with a lack of tetrahydrobiopterine were treated with a co-factor. In an evaluation of the reliability or confidence several dose dependent undesired side effects were observed, such as interference with sleep, polyurea and thin stool (BIOPTEN® licensure ticket (Approbationszettel), Suntory, Japan).

Several interferences must be cleared out of the way, before the treatment with tetrahydrobiopterine can become a routine treatment. First in most countries tetrahydrobiopterine is not an approved medicament. Second it is expensive. Third there is still a need for studies regarding the doses to be administered, as well as clinical research with regard to the bioavailability and the still unknown longtime side effects of tetrahydrobiopterine in phenylalanine hydroxylase deficiency.

In conclusion, it can be said that we have shown that pharmacological doses of tetrahydrobiopterine in most patients with hyperphenylalaninamie of a less heavy phenotype can be significantly improved or even normalized via a curing or elimination of protein misfolding interfered with phenylalanine oxidation. Beyond this, an improved protein tolerance and a relaxation of the dietetic measures can be achieved. This recognition is of importance for the diagnostic procedure, the clinical classification and the therapeutic process. In the near future the co-factor treatment will free many patients of a very burdensome restriction of the diet.

TABLE 1
Genotypes of Patients with Tetrahydrobiopterine-
Sensitive and Not-Sensitive Hyperphenylalaninamie

| | | | | TETRAHYDRO- |
|---|---|---|---|---|
| | | | PHENO- | BIOPTERINE- |
| ID | ALLELE 1 | ALLELE 2 | TYPE | SENSITIVITY |
| 1 | A403V | IVS4 + 5G > T | Mild | Yes |
| 2 | A403V | n.i. | Mild | Yes |
| 3 | P314S* | R408W⁺ | Mild | Yes |
| 4 | F39L | D415N | Mild | Yes |
| 5 | Y414C | D415N | Mild | Yes |
| 6 | Y417H* | Y417H* | Mild | Yes |
| | | | Phenyl- | |
| | | | ketonurea | |
| 7 | F55L | S310Y* | Mild | Yes |
| 8 | R261Q | Y414C | Mild | Yes |
| | | | Phenyl- | |
| | | | ketonurea | |
| 9 | V177M | R408W⁺ | Mild | Yes |
| 10 | P275L* | Y414C | Mild | Yes |
| | | | Phenyl- | |
| | | | ketonurea | |
| 11 | V245A | R408W⁺ | Mild | Yes |
| 12 | L48S | R158Q | Mild | Yes |
| | | | Phenyl- | |
| | | | ketonurea | |
| 13 | Y417H* | Y417H* | Mild | Yes |
| | | | Phenyl- | |
| | | | ketonurea | |
| 14 | V245A | R408W⁺ | Mild | Yes |
| 15 | R261X⁺ | A300S | Mild | Yes |
| | | | Phenyl- | |
| | | | ketonurea | |
| 16 | R158Q | E390G | Mild | Yes |
| | | | Phenyl- | |
| | | | ketonurea | |
| 17 | R261X⁺ | A300S | Mild | Yes |
| | | | Phenyl- | |
| | | | ketonurea | |
| 18 | Y414C | IVS12 + 1G > A⁺ | Mild | Yes |
| | | | Phenyl- | |
| | | | ketonurea | |
| 19 | I65S* | A300S | Mild | Yes |
| | | | Phenyl- | |
| | | | ketonurea | |
| 20 | R261Q | Y414C | Mild | Yes |
| | | | Phenyl- | |
| | | | ketonurea | |
| 21 | K274fsde111b | E390G | Mild | Yes |
| | | | Phenyl- | |
| | | | ketonurea | |
| 22 | IVS4 − 5C > G | R408W⁺ | Mild | Yes |
| | | | Phenyl- | |
| | | | ketonurea | |
| 23 | R261X⁺ | A300S | Mild | Yes |
| | | | Phenyl- | |
| | | | ketonurea | |
| 24 | I65T | Y414C | Mild | Moderate |
| | | | Phenyl- | |
| | | | ketonurea | |
| 25 | E390G | IVS12 + 1G > A⁺ | Mild | Moderate |
| | | | Phenyl- | |
| | | | ketonurea | |
| 26 | I65V | R261Q | Mild | Moderate |
| 27 | R158Q | Y414C | Mild | Moderate |
| | | | Phenyl- | |
| | | | ketonurea | |
| 28 | Y414C | IVS12 + 1G > A⁺ | Classic | No |
| 29 | P281L⁺ | Y414C | Mild | No |
| | | | Phenyl- | |
| | | | ketonurea | |
| 30 | I65V | IVS12 + 1G > A⁺ | Mild | No |
| | | | Phenyl- | |
| | | | ketonurea | |
| 31 | 165V | IVS12 + 1G > A⁺ | Mild | No |
| | | | Phenyl- | |
| | | | ketonurea | |
| 32 | N61D* | R261Q | Mild | No |
| | | | Phenyl- | |
| | | | ketonurea | |
| 33 | R408W⁺, | Y414C | Classic | No |
| | R413P | | | |
| 34 | P281L⁺ | P281L⁺ | Classic | No |
| 35 | R243X⁺ | Y414C | Classic | No |
| 36 | L48S | P281L⁺ | Classic | No |
| 37 | R261Q | R408W⁺ | Classic | No |
| 38 | R243X⁺ | IVS7 + 1G > A | Classic | No |

Mutations which with high probability are associated with tetrahydrobiopterine sensitivity are shown in grey.
Mutations which are potentially associated with tetrahydrobiopterine sensitivity are shown in bold.
Mutations of which the association with tetrahydrobiopterine sensitivity is inconsistent or inconclusive are shown in italics.
*Previously Undescribed Mutation
⁺Putative Mutation
n.i.—Not Identified

### FIGURE LEGEND

### FIG. 1

The effect of tetrahydrobiopterine on the phenylalanine concentration in blood. Phenylalanine concentration in blood (Phe) prior to the phenylalanine exposure and prior and subsequent to the provocation with tetrahydrobiopterine (BH₄). The boxes represent the 50% reliability interval (25-75 percentile); the horizontal black bars represent the median; the error bar shows the distance between minimum and maximum. The value P concerns the difference between the phenylalanine content in blood prior to and 15 hours subsequent to the administration of tetrahydrobiopterine.

### FIG. 2

The effect of short time treatment with tetrahydrobiopterine on the phenylalanine oxidation in vivo. A cumulative ¹³CO₂ (180 min.)—recapture prior to and subsequent to the treatment with tetrahydrobiopterine (BH₄). The boxes represent the 50% confidence interval (25-75 percentile); the horizontal black bars represent the median; the error bar shows the distance between minimum and maximum. B-E Fraction analysis of the ¹³CO₂ formation in representative patients with an impaired phenylalanine hydroxylase prior to (o) and subsequent to (λ) a short time treatment with tetrahydrobiopterine.

### FIG. 3

Relationship between the cumulative ¹³CO₂ recapture (180 min.) and the phenylalanine concentration in blood prior to and subsequent to the treatment with tetrahydrobiopterine (BH₄). Patients, which did not respond to tetrahydrobiopterine: 0; patients, which responded to tetrahydrobiopterine: λ; patients, which had a moderate response to tetrahydrobiopterine: λ.

### FIG. 4

Effect of tetrahydrobiopterine on the peripheral phenylalanine clearance and on the oxidation rate in individual hyperphenylalaninamie patients. The phenylalaninamie concentration in blood prior to (solid bar) and 15 hours after the administration of tetrahydrobiopterine (BH4) (dark gray bar). The positive effect obtained by tetrahydrobiopterine in individual patients are shown by a black arrow (upper field). Cumulative ¹³CO₂ recapture (180 min.) prior to (light gray bar) and subsequent to the administration of tetrahydrobiopterine (solid bar). The improvement caused by tetrahydrobiopterine in individual patients is represented by a dark arrow (lower field). The normal range (n. r.) for the in vivo phenylalanine oxidation, which was observed by a healthy controlled group in the age of 2 days to 13 years is indicated or shown (8.3±2.8%; average±SD, n=12). Irregularities in the effect of tetrahydrobiopterine: clear lowering of the phenylalanine concentration in blood, however slight elevation of the phenylalanine oxidation in one patient (λ) and small effect on the phenylalanine concentration in blood as well as a large increase in phenylalanine oxidation in a different patient (H). Slight response to tetrahydrobiopterine did not correspond to the criteria of the responsiveness to tetrahydrobiopterine in a patient with classical phenylketonurea (v).

### FIG. 5

Structural localization of phenylalanine hydroxylase missense mutation. The phenylalanine-hydroxylase-monomer, shown in the form of a band, is comprised of three functional domains: The regulator domain (Sequences 1-142), the catalytic domain (Sequences 143-410) and the tetramerization domain (Sequence 411-452). The iron at the active center (brown area, partially covered) and the co-factor analog 7,8-dihydro-tetrahydrobiopterine stick model is on the catalytic domain. Mutations, which are associated with the response to tetrahydrobiopterine with high probability, are shown in turquoise. Mutations, which possibly are connected with the response to tetrahydrobiopterine are shown in green. Mutations which inconsistently correspond with the response to tetrahydrobiopterine are shown in purple.

1-44. (canceled)

45. A method for long term treatment of conditions of reduced protein tolerance due to reduced phenylalanine oxidation without deficiency of cofactor tetrahydrobiopterine, said conditions caused by mutations in the phenylalanine hydroxylase gene associated with at least one of the following allele pairs:
A403V+IVS4+5G>T, P314S+R408W, F39L+D415N, Y414C+D415N, Y417H+Y417H, F55L+S310Y, V177M+R408W, P275L+Y414C, V245A+R408W, L48S+R158Q, Y417H+Y417H, V245A+R408W, R261X+A300S, R158Q+E390G, Y414C+IVS12+1G>A, I65S+A300S, H170O+A300S, R261Q+Y414C, K274fsdel11bp+E390G, IVS4-5C>G+R408W, I65T+Y414C, E390G+IVS12+1G>A, I65V+R261Q, R158Q+Y414C.
said method comprising administering a medicament containing at least one compound with the following general formula: 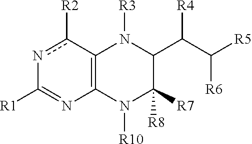
wherein R1 is selected from the group consisting of: H, OH, SH, F, Cl, Br, I, NH₂, N(CH₃)₂, N(C₂H₅)₂, N(C₃H₇)₂; NH-acyl, wherein the acyl residue contains 1 to 32 carbon atoms;
wherein R2 is selected from the group consisting of: H, OH, SH, NH₂, F, Cl, Br, I, O, S;
wherein R3 is selected from the group consisting of: H, CH₃, C₂H₅;
wherein R4 and R6 are selected independently of each other from the group consisting of: H, OH, SH, NH₂, F, Cl, Br, I, acetyl, OX, wherein X is a C1 to C32 acyl residue;
wherein R5 is selected from the group consisting of: phenyl, CH₃, C₂H₅, C₃H₇, butyl, isobutyl, t-butyl;
wherein R7 and R8 are selected independently of each other from the group consisting of: H, OH, SH, NH₂, F, Cl, Br, I, CH₃, COOH, CHO, COOR9, wherein R9 is CH₃, C₂H₅, C₃H₇, or butyl;
wherein R10 is selected from the group consisting of: H, CH₃, C₂H₅, and -- represents an optional double bond;
as well as their pharmaceutically acceptable salts.

46. A method as in claim 45, wherein said medicament is administered to a patient in need thereof until said patient exhibits improvement in protein tolerance.

47. A method as in claim 45, wherein R1 is NH-acyl, wherein the acyl residue contains CH₃O or 9 to 32 carbon atoms, and wherein at least one of R4 and R6 are C9 to C32 acyl residues.

48. A method according to claim 45, wherein the compound is selected from the group consisting of: 5,6,7,8-tetrahydrobiopterin, sapropterin, a compound having the following structure: 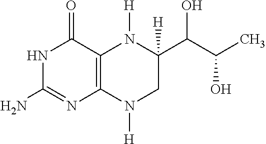 [SMILES: C[C@H](O)C(O)[C@H]1CNc2nc(N)[nH]c(=O)c2N1]
(−)-(1′R,2'S,6R)-2-amino-6-(1′,2′-dihydroxypropyl)-5,6,7,8-tetrahydro-4(3H)-pteridinone,
and/or
2-N-stearoyl-1′,2′-di-O-acetyl-5,6,7,8-tetrahydrobiopterin; and/or
2-N-decanoyl-1′,2′-di-O-acetyl-5,6,7,8-tetrahydrobiopterin; and/or
2-N-palmitoyl-1′,2′-di-O-acetyl-5,6,7,8-tetrahydrobiopterin; and/or
2-N-linoleoyl-1′,2′-di-O-acetyl-5,6,7,8-tetrahydrobiopterin.

49. A method according to claim 45, wherein said pharmaceutically acceptable salt a hydrochloride or a sulphate.

50. A method according to claim 45, wherein the condition of reduced protein tolerance is at least one of: conditions with increased phenylalanine or reduced tyrosine in body fluids, tissues or cells.

51. A method as in claim 50, wherein said condition of reduced protein tolerance is classical phenylketonurea, mild phenylketonurea or mild hyperphenylalaninemia

52. A method according to claim 45, wherein said medicament functions as chaperone for improving protein folding, in particular in the case of structural anomalies of enzymes, which require tetrahydrobiopterine as a cofactor.

53. A method according to claim 52, wherein said enzyme is selected from phenylalanine hydroxylase, tyrosine hydroxylase, tryptophan hydroxylase, or NO-synthase.

54. A method according to claim 45, wherein said compound functions as chaperone as neurotransmitter and/or second messenger enhancer, in particular in conditions with increased phenylalanine or lowered tyrosine, serotonin or dopamine in body fluids, tissues or cells, in particular in conditions with reduced phenylalanine hydroxylase, tyrosinhydroxylase, tryptophanhydroxylase, or NO-synthase activity.

55. A method according to claim 45, wherein said compound functions as neurotransmitter or as second messenger enhancer, in particular for catecholamine and/or serotonin and/or dopamine and/or nitrogen oxide (NO).

56. A composition comprising
(a) at least one compound with the following general formula: 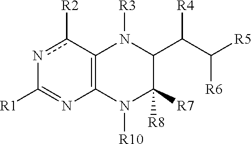
wherein R1 is selected from the group consisting of: H, OH, SH, F, Cl, Br, I, NH₂, N(CH₃)₂, N(C₂H₅)₂, N(C₃H₇)₂; NH-Acyl, wherein the Acyl residue contains 1 to 32 carbon atoms;
wherein R2 is selected from the group consisting of: H, OH, SH, NH₂, F, Cl, Br, I, O, S;
wherein R3 is selected from the group consisting of: H, CH₃, C₂H₅;
wherein R4 and R6 are selected independently of each other from the group consisting of: H, OH, SH, NH₂, F, Cl, Br, I, Acetyl, OX, wherein X is a C1 to C32 acyl residue;
wherein R5 is selected from the group consisting of: phenyl, CH₃, C₂H₅, C₃H₇, butyl, isobutyl, t-butyl;
wherein R7 and R8 are selected independently of each other from the group consisting of: H, OH, SH, NH₂, F, Cl, Br, I, CH₃, COOH, CHO, COOR9, wherein R9 is CH₃, C₂H₅, C₃H₇, or butyl;
wherein R10 is selected from the group consisting of: H, CH₃, C₂H₅, and - - represents an optional double bond;
as well as their pharmaceutically acceptable salts, as well as
(b) at least one amino acid selected from the group consisting of
essential amino acids: isoleucine, leucine, lysine, methionine, threonine, tryptophane, valine, histidine; as well as from
the non-essential amino acids, in particular alanine, arginine, asparaginic acid, asparagine, cysteine, in particular acetylcysteine, glutamic acid, glutamine, clycine, proline, serine as well as tyrosine,
wherein the following compounds are excluded: 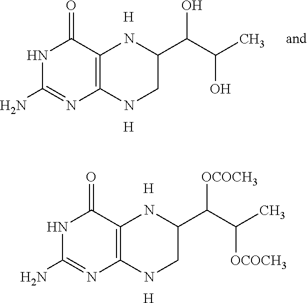
in the case that the amino acid is one of tryptophan, cysteine, in particular acetylcysteine, and tyrosine.

57. A composition according to claim 56, wherein the essential amino acids are selected from the group consisting of isoleucine, leucine, lysine, methionine, threonine, tryptophan, valine, histidine; and that it further contains at least one of the following amino acids: alanine, arginine, asparaginic acid, asparagine, cysteine, in particular acetylcysteine, glutamic acid, glutamine, clycine, proline, serine as well as tyrosine.

58. A composition according to claim 56, further comprising a hydrocarbon, in particular glucose, and/or vitamins.

59. A composition according to claim 56, formulated as an oral or intravenous preparation.

60. A composition according to claim 59, wherein said composition is in the form of a powder, tablet, capsule, pill, droplets, or as solution for IV administration.

61. A composition according to claim 56, in the form of a pharmaceutical preparation, optionally with pharmaceutical adjuvants or excipients.

62. A composition according to claim 56, wherein the compound is selected from the group consisting of: sapropterin, in particular the hydrochloride thereof, as well as a compound with the following structure: 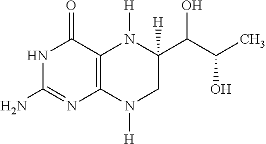 [SMILES: C[C@H](O)C(O)[C@H]1CNc2nc(N)[nH]c(=O)c2N1]
(−)-(1′R,2'S,6R)-2-amino-6-(1′,2′-dihydroxypropyl)-5,6,7,8-tetrahydro-4(3H)-pteridinone, in particular the dihydrochloride or sulphate thereof, and/or
2-N-stearoyl-1′,2′-di-O-acetyl-5,6,7,8-tetrahydrobiopterin; and/or
2-N-decanoyl-1′,2′-di-O-acetyl-5,6,7,8-tetrahydrobiopterin; and/or
2-N-palmitoyl-1′,2′-di-O-acetyl-5,6,7,8-tetrahydrobiopterin; and/or
2-N-linoleoyl-1′,2′-di-O-acetyl-5,6,7,8-tetrahydrobiopterin.

63. A special food based on mixtures of essentially phenylalanine-free mixtures, wherein it contains at least one compound of the following general formula: 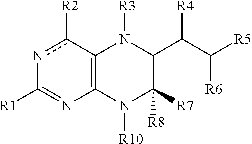
wherein R1 is selected from the group consisting of: H, OH, SH, F, Cl, Br, I, NH₂, N(CH₃)₂, N(C₂H₅)₂, N(C₃H₇)₂; NH-Acyl, wherein the Acyl residue contains 1 to 32 carbon atoms;
wherein R2 is selected from the group consisting of: H, OH, SH, NH₂, F, Cl, Br, I, O, S;
wherein R3 is selected from the group consisting of: H, CH₃, C₂H₅;
wherein R4 and R6 are selected independently of each other from the group consisting of: H, OH, SH, NH₂, F, Cl, Br, I, Acetyl, OX, wherein X is a C1 to C32 acyl residue;
wherein R5 is selected from the group consisting of: phenyl, CH₃, C₂H₅, C₃H₇, butyl, isobutyl, t-butyl;
wherein R7 and R8 are selected independently of each other from the group consisting of: H, OH, SH, NH₂, F, Cl, Br, I, CH₃, COOH, CHO, COOR9, wherein R9 is CH₃, C₂H₅, C₃H₇, or butyl;
wherein R10 is selected from the group consisting of: H, CH₃, C₂H₅, and - - represents an optional double bond;
as well as their pharmaceutically acceptable salts.

64. The special food of claim 62 wherein it contains at least one compound selected from the group consisting of: 5,6,7,8-tetrahydrobiopterin, sapropterin, in particular the hydrochloride thereof, as well as a compound with the following structure: 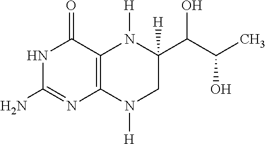 [SMILES: C[C@H](O)C(O)[C@H]1CNc2nc(N)[nH]c(=O)c2N1]
(−)-(1′R,2'S,6R)-2-amino-6-(1′,2′-dihydroxypropyl)-5,6,7,8-tetrahydro-4(3H)-pteridinone, in particular the dihydrochloride or sulphate thereof, and/or
2-N-stearoyl-1′,2′-di-O-acetyl-5,6,7,8-tetrahydrobiopterin; and/or
2-N-decanoyl-1′,2′-di-O-acetyl-5,6,7,8-tetrahydrobiopterin; and/or
2-N-palmitoyl-1′,2′-di-O-acetyl-5,6,7,8-tetrahydrobiopterin; and/or
2-N-linoleoyl-1′,2′-di-O-acetyl-5,6,7,8-tetrahydrobiopterin.
